(19) United States
(12) Patent Application Publication
(10) Pub. No.: US 2026/0264247 A1
(43) Pub. Date: Sep. 10, 2026

(54) METHOD, PROGRAM, AND DEVICE FOR MONITORING CONTROL OF MEDICAL ROBOT

(71) Applicant: AIRS MEDICAL INC., Seoul (KR)

(72) Inventors: Yeongjae CHOI, Seoul (KR); Sangmin LEE, Gyeonggi-do (KR)

(21) Appl. No.: 18/866,046

(22) PCT Filed: Jul. 6, 2023
(86) PCT No.: PCT/KR2023/009560

§ 371 Date: May 20, 2026

(30) Foreign Application Priority Data
Jul. 6, 2022 (KR) 10-2022-0083070

(51) Int. Cl. B25J 9/16; A61B 34/20; A61B 34/30; B25J 13/08; G06T 7/246; G06T 7/73

(52) U.S. Cl. CPC B25J 9/1692; A61B 34/30; B25J 9/1697; B25J 13/089; G06T 7/248; G06T 7/74; A61B 2034/2055; A61B 2034/2065; G06T 2207/30004; G06T 2207/30204

(57) ABSTRACT

There are disclosed a method, program and device for monitoring the control of a medical robot according to an embodiment of the present disclosure. The method may include: generating first position data indicating the relative positions between two or more parts included in a medical robot based on an image acquired through a camera; generating second position data indicating the relative positions between the parts based on control data intended to move the parts to target positions; computing an error between the first position data and the second position data; and monitoring the results of movements of the parts to the target positions based on the error.

### TECHNICAL FIELD

The present disclosure relates to a method and device for monitoring the control of a medical robot, and more particularly, to a method and device for monitoring the control of movement of parts by utilizing the images acquired by photographing parts included in a medical robot and the internal control information related to the parts included in the medical robot.

### BACKGROUND ART

As robotics technology innovation expands the use of the robotics technology in various medical fields such as surgery, research and treatment, the development of medical robots is being actively performed. Due to the nature of the medical field, medical robots require high accuracy in their movements made to perform functions such as surgery, rehabilitation assistance, etc. Accordingly, it is important to design medical robots to perform specific movements accurately. However, it is difficult to design medical robots to perform movements without errors in reality, so that it is also important to check whether medical robots perform specific movements accurately and to accurately calibrate the medical robots before functions according to the specific movements are performed.

A specific movement of a medical robot is implemented through the movement of a plurality of parts included in the medical robot. Accordingly, in order to verify and calibrate the accuracy of a specific movement of a medical robot, there have been developed technologies that guarantee positional accuracy according to the movement of each of a plurality of parts included in the medical robot. For example, one of the conventional technologies determines whether individual parts have moved to an exact target position by analyzing the positional relationship in such a manner as to connect the movement points of the individual parts from a reference point where positional accuracy is guaranteed. Another of the conventional technologies determines the positional accuracy of a plurality of parts by analyzing the positional relationship based on the origin after sequentially moving the plurality of parts on a per-control unit basis. However, these conventional technologies have technical limitations in that they may not be implemented under specific conditions such as a case where there is a constraint on movement due to the design of a medical robot and a case where a plurality of parts included in a medical robot are controlled simultaneously.

### DISCLOSURE

### Technical Problem

The present disclosure has been conceived in response to the aforementioned background technology, and an object of the present disclosure is to provide a monitoring method and device that enable the precise movement control and calibration of parts included in a medical robot even under specific conditions such as a case where there is a restraint on movement due to the design of the medical robot and a case where a plurality of parts included in the medical robot are controlled simultaneously.

However, the objects to be accomplished by the present disclosure are not limited to the object mentioned above, and other objects not mentioned may be clearly understood based on the following description.

### Technical Solution

According to one embodiment of the present disclosure for achieving the above-described object, there is disclosed a method for monitoring the control of a medical robot that is performed by a computing device including at least one processor. The method may include: generating first position data indicating the relative positions between two or more parts included in a medical robot based on an image acquired through a camera; generating second position data indicating the relative positions between the parts based on control data intended to move the parts to target positions; computing an error between the first position data and the second position data; and monitoring the results of movements of the parts to the target positions based on the error.

Alternatively, generating the first position data indicating the relative positions between the two or more parts included in the medical robot based on the image acquired through the camera may include: estimating feature points for the parts based on the image acquired through the camera; and generating the first position data based on the estimated feature points.

Alternatively, the feature points may be present on a same plane of the image and constitute three or more pairs.

Alternatively, estimating the feature points for the parts based on the image acquired through the camera may include: estimating first feature points for the parts based on a first image acquired through the camera before the parts move to the target positions; and estimating second feature points for the parts based on a second image acquired through the camera after the parts have moved to the target positions.

Alternatively, generating the first position data based on the estimated feature points may include: matching the first feature points against the second feature points; and generating position data indicating the relative positions between the parts after the parts have moved to the target positions based on the results of the matching.

Alternatively, generating the position data indicating the relative positions between the parts after the parts have moved to the target positions based on the results of the matching may include: estimating a matrix for the geometric transformation of the first and second images using the results of the matching; and estimating the coordinate differences between the parts after the parts have moved to the target positions based on the matrix.

Alternatively, the method may further include determining whether markers marked on the parts are present based on the image acquired through the camera.

Alternatively, when it is determined that markers marked on the parts are present, the feature points may correspond to the markers.

Alternatively, when it is determined that the markers marked on the parts are not present, estimating the feature points for the parts based on the image acquired through the camera may include: extracting the feature points from the image; and computing feature descriptors based on the feature points.

Alternatively, when it is determined that the markers marked on the parts are not present, generating the first position data based on the estimated feature points may include: matching first feature points extracted based on a time point before the parts move to the target positions against second feature points extracted based on a time point after the parts have moved to the target positions based on the feature descriptors.

Alternatively, the control data may include at least one of coordinate values generated by the processor of the medical robot to move the parts to the target positions, sensor values adapted for the detection of positions of the parts, and encoder values of a motor intended for the movements of the parts.

Alternatively, monitoring the results of the movements of the parts to the target positions based on the error may include estimating the positional accuracy of the parts for the target positions by comparing the error with a reference value.

Alternatively, monitoring the results of the movements of the parts to the target positions based on the error may include determining whether calibration is required for at least one of the positions of the parts by comparing the error with a reference value.

Alternatively, monitoring the results of the movements of the parts to the target positions based on the error may include, when it is determined that the error exceeds the reference value, determining whether the control of at least one of the parts requiring calibration is possible.

Alternatively, monitoring the results of the movements of the parts to the target positions based on the error may include, when it is determined that the control of at least one of the parts requiring calibration is impossible or that the error cannot be calibrated within a reference value through control, generating an alarm indicating that the calibration is required.

According to one embodiment of the present disclosure for achieving the above-described object, there is disclosed a computer program stored in a computer-readable storage medium. The computer program performs operations for monitoring the control of a medical robot when executed on at least one processor. In this case, the operations may include the operations of: generating first position data indicating the relative positions between two or more parts included in a medical robot based on an image acquired through a camera; generating second position data indicating the relative positions between the parts based on control data intended to move the parts to target positions; computing an error between the first position data and the second position data; and monitoring the results of movements of the parts to the target positions based on the error.

According to one embodiment of the present disclosure for achieving the above-described object, there is disclosed a computing device for monitoring the control of a medical robot. The computing device may include: a processor including at least one core; memory including program codes executable on the processor; and a camera configured to photograph two or more parts included in a medical robot. In this case, the processor may generate first position data indicating the relative positions between two or more parts included in a medical robot based on an image acquired through a camera, may generate second position data indicating the relative positions between the parts based on control data intended to move the parts to target positions; may compute an error between the first position data and the second position data; and may monitor the results of movements of the parts to the target positions based on the error.

### Advantageous Effects

The method and device according to the present disclosure may increase the accuracy of position control and convenience of control calibration for a plurality of parts included in a medical robot.

### DESCRIPTION OF DRAWINGS

FIG. 1 is a block diagram of a computing device according to one embodiment of the present disclosure;

FIG. 2 is a conceptual diagram illustrating a process of performing monitoring through a computing device according to the one embodiment of the present disclosure;

FIG. 3 is a flowchart illustrating a method for monitoring the control of a medical robot according to one embodiment of the present disclosure;

FIG. 4 is a flowchart showing a process of monitoring the control of a medical robot according to an embodiment of the present disclosure; and

FIG. 5 is a flowchart illustrating a part of a computation process for monitoring the control of a medical robot according to one embodiment of the present disclosure.

### MODE FOR INVENTION

Embodiments of the present disclosure will be described in detail below with reference to the accompanying drawings so that those having ordinary skill in the art of the present disclosure (hereinafter referred to as those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings. The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “N-th (N is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution n of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “model” used herein may be understood as a system implemented using mathematical concepts and language to solve a specific problem, a set of software units intended to solve a specific problem, or an abstract model for a process intended to solve a specific problem. For example, a neural network “model” may refer to an overall system implemented as a neural network that is provided with problem-solving capabilities through training. In this case, the neural network may be provided with problem-solving capabilities by optimizing parameters connecting nodes or neurons through training. The neural network “model” may include a single neural network, or may include a neural network set in which multiple neural networks are combined together.

The term “image” used herein may refer to multidimensional data composed of discrete image elements. In other words, the “image” may be understood as a term referring to a digital representation of an object that can be seen by the human eye. For example, the term “image” may refer to multidimensional data composed of elements corresponding to pixels in a two-dimensional image. The term “image” may refer to multidimensional data composed of elements corresponding to voxels in a three-dimensional image.

The term “medical robot” of the present disclosure may collectively refer to robots that support or assist all medical procedures performed in the medical field. Furthermore, the “part” included in the “medical robot” of the present disclosure may be understood as a component unit that is controlled by the robot to perform a specific function or task. In other words, the “part” included in the “medical robot” may be a component of the robot whose movement is controlled by a processor, included in the “medical robot,” for a specific function or task. For example, the “medical robot” may include a venipuncture robot that includes a blood collection or intravenous (IV) injection function for the diagnosis of diseases, a blood transfusion, and/or the like. In this case, the “part” included in the “medical robot” may include an end-effector required for blood collection or intravenous injection, and may be a component that implements a movement for blood collection or intravenous injection in a three-dimensional space. The “medical robot” is not limited to the examples described above, and may include robots that assist medical personnel in performing surgical operations at surgical operation sites.

The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

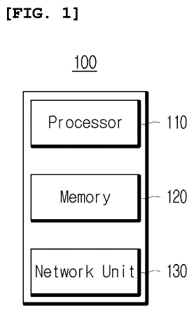

FIG. 1 is a block diagram of a computing device according to one embodiment of the present disclosure.

The computing device 100 according to the one embodiment of the present disclosure may be a hardware device or a part of a hardware device that performs the comprehensive processing and computation of data, or may be a software-based computing environment that is connected to a communication network. For example, the computing device 100 may be a server that is a principal agent performing an intensive data processing function and sharing resources, or may be a client that shares resources through interaction with a server or a specific terminal. Furthermore, the computing device 100 may be a cloud system in which pluralities of servers and clients comprehensively process data while interacting with each other. Furthermore, the computing device 100 may be a component of a medical robot. Since the above-described description is only one example related to the type of the computing device 100, the type of the computing device 100 may be configured in various manners within a range understandable to those skilled in the art based on the content of the present disclosure.

Referring to FIG. 1, the computing device 100 according to the one embodiment of the present disclosure may include a processor 110, memory 120, and a camera 130. However, FIG. 1 shows only an example, and the computing device 100 may include other components for implementing a computing environment. Alternatively, only some of the components disclosed above may be included in the computing device 100.

The processor 110 according to one embodiment of the present disclosure may be understood as a component unit including hardware and/or software for performing computing operations. For example, the processor 110 may read a computer program and perform data processing for the control of the robot. The processor 110 may process computational processes such as processing data for the monitoring of the operation of the robot, the computation of errors, etc. The processor 110 for performing such data processing may include a central processing unit (CPU), a general purpose graphics processing unit (GPGPU), a tensor processing unit (TPU), an application specific integrated circuit (ASIC), or a field programmable gate array (FPGA). Since the types of processor 110 described above are only examples, the type of processor 110 may be configured in various manners within a range understandable to those skilled in the art based on the content of the present disclosure.

The processor 110 can monitor the states of a plurality of parts included in the medical robot before and after movement based on the images acquired through the camera 130 and the control data for the parts included in the medical robot. In this case, the control data may include internal data that can be acquired in the process in which the medical robot controls the movement of the parts. For example, the control data may include at least one of control information generated to control the movement of the parts and mechanical information acquired as the medical robot controls the movement of the parts. In the process in which the medical robot controls the movement of a plurality of parts to perform a specific function, the processor 110 may determine whether the parts have accurately reached target positions based on a time point at which the parts have moved. When the parts have not accurately moved to the target positions, the processor 110 may directly perform calibration for the position of at least one of the parts, or may generate a calibration signal that allows the medical robot to perform calibration on its own. Furthermore, the processor 110 may generate a system alarm providing notification that calibration is required for the position of at least one of the parts.

More specifically, the processor 110 may estimate the relative positions between the plurality of parts included in the medical robot by using the images acquired as the camera 130 photographs the parts and the control data generated in the process of controlling the parts of the medical robot. In this case, the relative positions may be understood as indicating the results of comparing where the plurality of parts are located in space based on the images and the control data, rather than indicating where the individual parts are located relative to a specific reference position. Based on the estimated relative positions, the processor 110 may compute the error between the actual control results (i.e., observation results after control) identified through the images and the intended control results of the medical robot identified through the control data. Based on the error, the processor 110 may estimate the positional accuracy indicating how accurately the parts are located at the target positions. Furthermore, the processor 110 may determine whether calibration is required for at least one of the parts by comparing the error with a reference value. When it is determined that calibration is required, the processor 110 may generate a calibration signal so that the medical robot can perform the calibration on its own, or may perform the calibration by directly controlling the part to be calibrated. When the control of the part to be calibrated cannot be performed by the medical robot itself, or when it is impossible to calibrate the part to be calibrated within an error range even when the control of the part to be calibrated is performed, the processor 110 may generate an alarm providing notification that the part to be calibrated requires calibration. In this case, the alarm generated by the processor 110 may be notified to a user through a separate client or the like.

In this manner, the monitoring of the processor 110 may be performed based on the relative positions of the plurality of parts included in the medical robot. Accordingly, even when a plurality of parts are simultaneously controlled and moved, the processor 110 may monitor control results by rapidly and accurately computing the error between actual values and control values, and may allow additional calibration to be performed accurately and conveniently.

The memory 120 according to one embodiment of the present disclosure may be understood as a component unit including hardware and/or software for storing and managing data that is processed in the computing device 100. That is, the memory 120 may store any type of data generated or determined by the processor 110 and any type of data received over a communication network. For example, the memory 120 may include at least one type of storage medium out of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 120 may include a database system that controls and manages data in a predetermined system. Since the types of memory 120 described above are only examples, the type of memory 120 may be configured in various manners within a range understandable to those skilled in the art based on the content of the present disclosure.

The memory 120 may structure, organize, and manage data required for the processor 110 to perform computation, the combinations of data, and program codes executable on the processor 110. For example, the memory 120 may store videos or images acquired through the camera 130, which will be described later. The memory 120 may store program codes that operate the processor 110 to process images, program codes that operate the processor 110 to perform error computation for the monitoring of the control of the medical robot, and various data generated as the program codes are executed.

The camera 130 according to one embodiment of the present disclosure may be a device for measuring the shapes of two or more parts, included in the medical robot, before and after movement. In order for the camera 130 to perform accurate measurement, it is important that the camera 130 is not affected by the movement or distortion of components that are controlled to allow the medical robot to perform a specific function. Accordingly, the camera 130 may be placed on an external frame outside the medical robot, or may be placed on a separate frame independent of the medical robot. The camera 130 placed on the external frame outside the medical robot or the separate frame independent of the medical robot may photograph the states of the parts before and after movement based on a series of time points at which the plurality of parts are controlled. In this case, the camera 130 may generate images before and after the series of time points at which the plurality of parts are controlled, or may generate images including the series of time points at which the plurality of parts are controlled. The images or videos generated by the camera 130 represent actual observation results, and may be utilized to compute the relative positions between the parts.

Although not disclosed in FIG. 1, the computing device 100 according to one embodiment of the present disclosure may further include a network unit. The network unit according to one embodiment of the present disclosure may be understood as a component unit that transmits and receives data through any type of known wired/wireless communication system. For example, the network unit may perform data transmission and reception using a wired/wireless communication system such as a local area network (LAN), a wideband code division multiple access (WCDMA) network, a long term evolution (LTE) network, the wireless broadband Internet (WiBro), a 5th generation mobile communication (5G) network, a ultra wide-band wireless communication network, a ZigBee network, a radio frequency (RF) communication network, a wireless LAN, a wireless fidelity network, a near field communication (NFC) network, or a Bluetooth network. Since the above-described communication systems are only examples, the wired/wireless communication system for the data transmission and reception of the network unit may be applied in various manners other than the above-described examples.

The network unit may receive data, required for the processor 110 to perform computation, through wired/wireless communication with the medical robot. Furthermore, the network unit may transmit data, generated through the computation of the processor 110, through wired/wireless communication with the medical robot. For example, the network unit may receive control data for the parts included in the medical robot through communication with the medical robot. The network unit may transmit calibration control signals, alarm signals, or the like obtained through the computation of the processor 110 through communication with the medical robot.

Meanwhile, the computing device 100 according to the one embodiment of the present disclosure may be an independent device that performs monitoring through network communication with the medical robot as shown in FIG. 1, or may be an on-device type component dependent on the medical robot. For example, when the computing device 100 is a device independent of the medical robot, the computing device 100 may be a cloud system that controls and monitors the movement of the parts, included in the medical robot, through cloud communication with the medical robot. When the computing device 100 is an on-device type component of the medical robot, the processor 110 and memory 120 of the computing device 100 may correspond to the processor and memory of the medical robot, respectively. Furthermore, the camera 130 of the computing device 100 may correspond to a component placed on the external frame outside the medical robot.

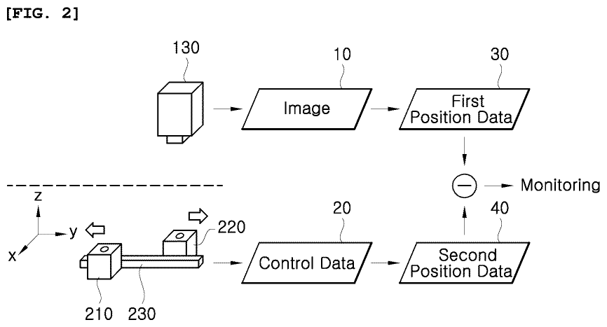

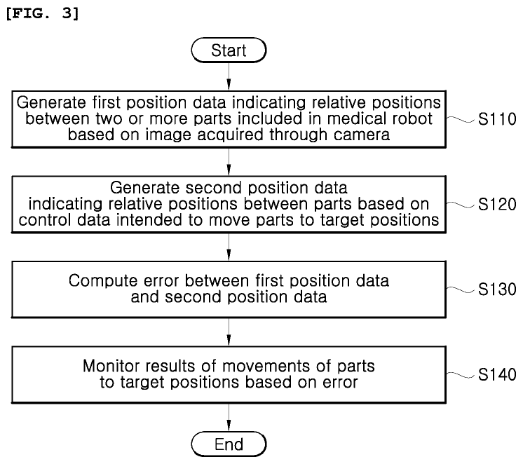

FIG. 2 is a conceptual diagram illustrating a process of performing monitoring through the computing device according to the one embodiment of the present disclosure, and FIG. 3 is a flowchart illustrating a method for monitoring the control of a medical robot according to one embodiment of the present disclosure.

Referring to FIGS. 2 and 3, the computing device 100 according to the one embodiment of the present disclosure may generate first position data 30 indicating the relative positions between two or more parts included in a medical robot based on an image 10 acquired through the camera 130 in step S110. When the image 10 of the parts is acquired through the camera 130 in the process in which movement control for the parts is performed, the computing device 100 may generate the first position data 30 indicating where the parts are relatively located before and after a specific time point based on the image 10. In this case, the first position data 30 may be understood as data regarding the relative positions between the two or more parts analyzed based on the results observed through the camera 130 that minimizes external influence occurring in the process of controlling the parts.

For example, the computing device 100 may estimate feature points for the parts based on the image 10 acquired through the camera 130 in the process in which the medical robot performs the movement control of the parts. In this case, the feature points are indicators for allowing the computing device 100 to recognize the parts shown in the image, and may be present on the same plane of the image. Furthermore, the feature points may be estimated in three or more pairs so that the relative positions between the parts can be precisely identified. Furthermore, the feature points may be distinguished based on the time points before and after the parts move to target positions through the control of the medical robot. The target positions may be final positions in the state in which the control of movement of the parts intended to perform a specific function is completed, or may be positions at a specific time point during the process of performing control for a specific function. That is, the computing device 100 may estimate first feature points for the parts based on a first image acquired through the camera 130 before the parts move to the target positions. Moreover, the computing device 100 may estimate second feature points for the parts based on a second image acquired through the camera 130 after the parts have moved to the target positions.

The computing device 100 may analyze where the parts are relatively located based on the above-described feature points and generate the first position data 30. More specifically, the computing device 100 may match the feature points that are distinguished based on the time points before and after the parts move to the target positions through the control of the medical robot. The computing device 100 may match the first feature points based on the time point before the parts move to the target positions against the second feature points based on the time point after the parts have moved to the target positions. This feature point matching may be understood as a computational process that allows the computing device 100 to recognize the parts, whose positions have been changed during a control process, on the image. The computing device 100 may generate position data indicating the relative positions between the parts after the parts have moved to the target positions based on the results of the matching between the first and second feature points. The computing device 100 may generate position data by analyzing the differences in positions after the two or more parts have moved to the target positions through the results of the matching between the first and second feature points. Accordingly, the first position data 30 may include position data, indicating the relative positions between the parts after the parts have moved to the target positions, that is generated based on the computation of the feature point matching of the image.

The computing device 100 according to the one embodiment of the present disclosure may generate second position data 40 indicating the relative positions between the two or more parts based on control data 20 intended to move the two or more parts to the target positions in step S120. When the control data 20 generated internally inside the medical robot in the process in which movement control for the parts is performed is acquired, the computing device 100 may generate the second position data 40, indicating where the parts are relatively located before and after a specific time point, based on the control data 20. In this case, the second position data 40 may be understood as data regarding the relative positions between the two or more parts analyzed based on data that can be acquired from the robot during the process of actually controlling the parts, such as the numerical information processed by the processor of the medical robot to control the parts, the mechanical information generated during the control process, and/or the like. For example, as shown in FIG. 2, it is assumed that the movements of two parts 210 and 220 equipped with end-effectors for the invasion of a venipuncture robot including a blood collection or venous injection function are controlled simultaneously. The computing device 100 may generate position data, indicating the relative positions between the two parts 210 and 220 after the two parts 210 and 220 have moved to target positions, based on control data 20 that can be acquired during the process of moving the two parts 210 and 220 laterally on the XY plane. The computing device 100 may generate position data indicating the relative positions between the two parts 210 and 220 in the state in which the movement control of the two parts 210 and 220 is completed by utilizing the numerical information intended to move the two parts 210 and 220 to the target positions, the mechanical information generated when the two parts 210 and 220 move to the target positions through a linker 230 that transmits the driving force of a motor, and/or the like. Accordingly, the second position data 40 may include position data, indicating the relative positions between the parts after the parts have moved to the target positions, that is generated based on the computation performed on the control data 20 that can be acquired throughout the overall control process for the two or more parts.

The control data 20 of the present disclosure may include at least one of coordinate values generated by the processor of the medical robot to move the plurality of parts to the target positions, sensor values adapted for the detection of the positions of the parts, and encoder values of the motor intended for the movements of the parts. More specifically, the computing device 100 may generate position data indicating the relative positions between the parts by using the coordinate values computed for the movement control of the two or more parts by the processor of the medical robot. The computing device 100 may generate position data indicating the relative positions between the parts by using information detected through sensors after the two or more parts have moved to the target positions. The computing device 100 may generate position data indicating the relative positions between the parts by using the encoder values of the motor driven in the process in which the two or more parts have moved to the target positions. However, as in the example described above, the computing device 100 may generate position data by combining the coordinate values, sensor values, and encoder values included in the control data 20, rather than separately using the coordinate values, the sensor values, or the encoder values.

The computing device 100 according to the one embodiment of the present disclosure may compute an error between the first position data 30 generated based on the image 10 and the second position data 40 generated based on the control data 20 in step S130. Since the first position data 30 and the second position data 40 represent the relative positions between two or more parts, they may be represented by the differences in the coordinate values that the two or more parts have in space. Accordingly, the computing device 100 may compute an error between the two pieces of data 30 and 40 based on the differences in coordinate values between the parts represented by the first position data 30 and the differences in coordinate values between the parts represented by the second position data 40. For example, in the state in which the two parts 210 and 220 have moved to the target positions through control, the computing device 100 may compute the extent of deviation present in the differences in the coordinate values represented by the two pieces of data 30 and 40 by comparing the differences in coordinate values between the two parts 210 and 220 represented by the first position data 30 generated based on the image 10 and the differences in coordinate values between the two parts 210 and 220 represented by the second position data 40 generated based on the control data 20 inside the robot. That is, the computing device 100 may compute an error between the differences in the relative positions between the two parts 210 and 220 observed through the camera 130 at the control point and the differences in the relative positions between the two parts 210 and 220 identified by the internal control information of the medical robot.

The computing device 100 may monitor the results of the movement of the two or more parts to the target positions based on the error between the first position data 30 and the second position data 40 in step S140. For example, the computing device 100 may estimate the position accuracies of the parts for the target positions by comparing the error between the first position data 30 and the second position data 40 with a reference value. In this case, the reference value may be a value pre-determined by the user, and may be modified through an external input applied through a client terminal or the like. More specifically, the reference value may be the maximum error set by the manufacturer of the medical robot to achieve the purpose of the medical robot according to the purpose of the medical robot. Since the error and reference value may vary depending on the importance of each part even in a single robot, the reference value may also be interpreted as a set of values that can be assigned to a plurality of parts, respectively. Furthermore, the positional accuracy may be an indicator indicating how much the control of each of the parts deviates from the value intended by the medical robot. The positional accuracy may be effectively utilized to check whether the medical robot is operating normally or to calibrate the position of each of the parts of the medical robot. The computing device 100 may determine whether calibration is required for the position of at least one of the two or more parts by comparing the error between the first position data 30 and the second position data 40 with the reference value. In other words, the computing device 100 may determine whether calibration is required for at least one of the two or more parts based on the comparison results. The computing device 100 may or may not perform direct calibration depending on the determination of whether calibration is required, and may also generate a separate alarm providing notification that calibration is required.

As described above, the computing device 100 may perform monitoring functions such as the function of checking whether the operation of the robot is normal and the function of calibrating the movement control of the parts by utilizing the relative positions between the two or more parts included in the medical robot. Since the computing device 100 utilizes the relative positions between the two or more parts included in the medical robot, it does not require a fixed mark or spatial information as a reference for the determination of positional accuracy unlike in the prior art. Accordingly, the computing device 100 of the present disclosure may be more effectively used for the control monitoring of a medical robot whose target position is not fixed and may change from time to time depending on the physical characteristics of a patient. For example, when the medical robot is a venipuncture robot including a blood collection or intravenous injection function, the position of blood vessel observation for invasion and invasion for blood collection or intravenous injection may continuously change depending on the physical characteristics of a patient, so that it is inevitably important to accurately identify the position immediately before the invasion according to the physical characteristics of the patient. The computing device 100 may accurately monitor the positions of the parts including an end-effector for invasion by analyzing the relative positions between the parts, rather than comparing the positions of the parts with target absolute positions, in accordance with this importance. Furthermore, since the computing device 100 analyzes the relative positions between the parts rather than comparing the positions of the parts with the target absolute positions, it may efficiently perform computation for monitoring even when two or more parts are controlled simultaneously, thereby minimizing the computational burden.

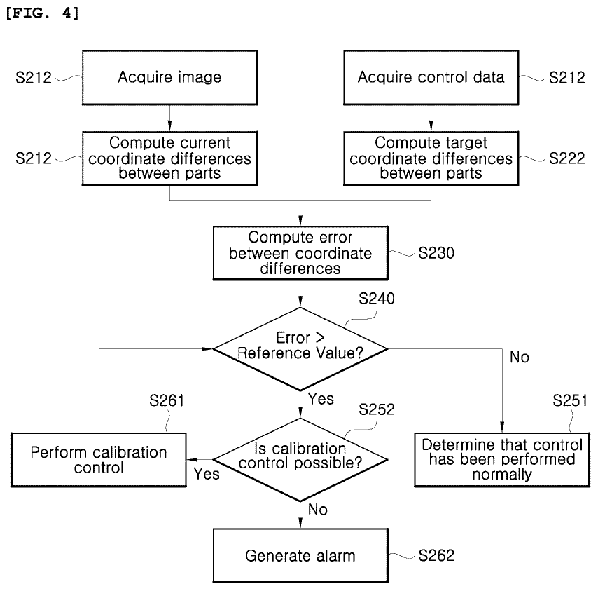

FIG. 4 is a flowchart showing a process of monitoring the control of a medical robot according to an embodiment of the present disclosure.

Referring to FIG. 4, the computing device 100 according to the one embodiment of the present disclosure may acquire an image of two or more parts included in a medical robot through the camera 130 in step S211. In this case, the image may be data generated after being photographed by the camera 130, or may be data extracted from an image photographed by the camera 130. When the movement control of the two or more parts included in the medical robot is performed, the computing device 100 may acquire an image of the parts through the camera 130 at a time point before the two or more parts move to target positions. The computing device 100 may acquire an image of the parts through the camera 130 at a time point after the two or more parts have moved to the target positions. That is, the computing device 100 may acquire images through the camera 130 before and after the two or more parts move to the target positions.

The computing device 100 may compute the differences in the current coordinates between the parts based on the images of the parts in step S221. In this case, the current coordinates may refer to the coordinates at a time point after the two or more parts have moved to the target positions through the control of the medical robot. That is, the computing device 100 may compute the differences in the coordinates between the parts at a time point after two or more parts have moved to the target positions by using the images before and after two or more parts move to the target positions. The differences in the coordinates between the parts may be understood as the results of comparing the positions of the two or more parts with each other, rather than the results of sequentially comparing the positions of the parts with an absolute reference position.

The computing device 100 may obtain control data for the two or more parts included in the medical robot in step S212. The control data may include numerical information generated by the medical robot to control the two or more parts, and/or mechanical information internally determined inside the medical robot during the process of controlling the two or more parts. In other words, in this case, the control data may be understood as internal information that can be obtained from the medical robot during the process of controlling the movement of the parts, rather than being derived from the results of observing the actual positions to which the parts have moved. When the movement control of the two or more parts included in the medical robot is performed, the computing device 100 may obtain control data such as a command of the processor to move the two or more parts to the target positions, an encoder value of the motor determined while moving the two or more parts to the target positions, and/or the like.

The computing device 100 may compute the differences in target coordinates between the parts based on the control data in step S221. The computing device 100 may compute the differences in coordinates between the parts at a time point after the two or more parts have moved to the target positions by using the control data. Accordingly, the differences in coordinates between the parts computed based on the control data may be understood as being derived based on the results of the arithmetic operations of numerical values included in the data, rather than being derived based on observation results such as images. However, like the differences in coordinates between the parts derived based on the images, the differences in coordinates between the parts based on the control data may also be understood as being the results of comparing the positions of the two or more parts with each other, rather than the results of sequentially comparing the positions of the parts with an absolute reference position.

In step S230, the computing device 100 may derive an error between the current coordinate differences between the parts computed through step S221 and the target coordinate differences between the parts computed through step S222. In this case, the error may be understood as an indicator indicating the extent of deviation in information about the relative positions by comparing the relative positions between the parts determined based on the images and the relative positions between the parts derived based on the control data.

The computing device 100 may monitor the results of the movement control of the parts by comparing the error derived through the step S230 with a reference value in step S240. In this case, the reference value may be a value determined in advance based on the theoretical error range within which the medical robot can normally perform a specific function or operation. When the error is determined to be equal to or smaller than the reference value, the computing device 100 may determine that the control of the robot has been performed normally in step S251. When it is determined that the control has been performed normally, the medical robot may perform additional control for a specific function or operation. In contrast, when the error is determined to exceed the reference value, the computing device 100 may determine which of the two or more parts is subject to calibration and also determine whether calibration is possible for at least one part determined to be subject to calibration in step S252.

When it is determined that calibration is possible for the at least one part, the computing device 100 may directly or indirectly perform control on the calibration target in step S261. For example, when the at least one part that is a calibration target can be moved to a calibration position through physical control in terms of the current design of the robot, the computing device 100 may determine that calibration is possible for the at least one part. Furthermore, the computing device 100 may perform control that directly calibrates the position of the at least one part that is a calibration target. The computing device 100 may also generate a calibration signal and transmit it to the processor of the medical robot so that the medical robot performs calibration for the at least one part that is a calibration target. When calibration for the at least one part is completed, the computing device 100 may monitor whether the positions of the parts are within a normal range by comparing the error with the reference value again.

When it is determined that calibration for the at least one part is impossible, the computing device 100 may generate an alarm indicating that calibration is required for the calibration target in step S261. For example, when the at least one part is structurally difficult to move to the calibration position due to the current design of the robot, the computing device 100 may determine that calibration for the at least one part is impossible. Furthermore, when the at least one part can move to the calibration position but it is impossible to calibrate the error within a reference value, the computing device 100 may determine that calibration for the at least one part is impossible. When calibration is impossible, the constraint on the movement of the part needs to be alleviated through a design change for the robot or the like. Accordingly, the computing device 100 may generate an alarm for the calibration target so that the user or the like can recognize the need for the alleviation of the restraint. In addition, the computing device 100 may notify the user of the alarm through communication with a client terminal or the like on the system.

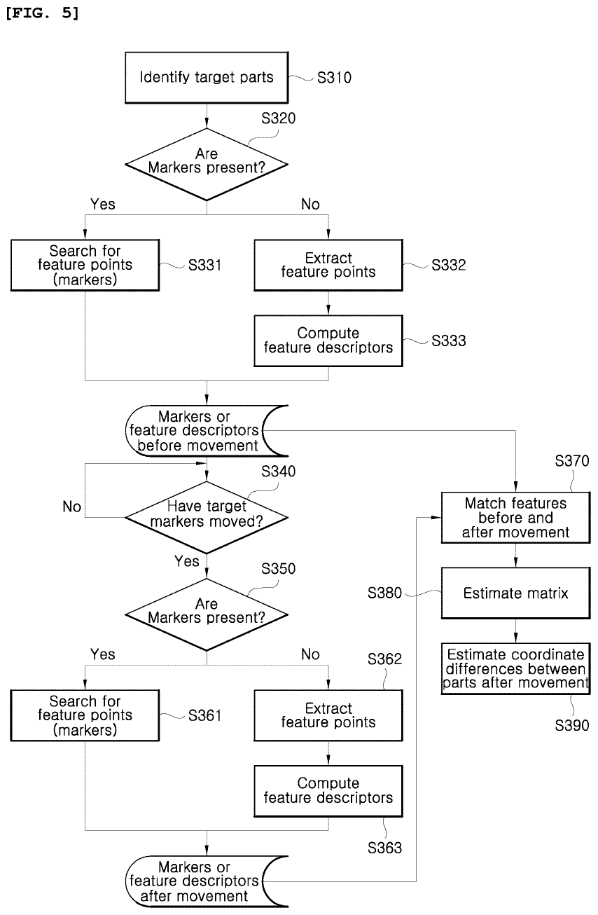

FIG. 5 is a flowchart showing a process in which a computing device according to an embodiment of the present disclosure determines relative positions between parts of a medical robot.

The processor 110 of the computing device 100 according to the one embodiment of the present disclosure may check the positional accuracy of the parts during the process in which the medical robot controls the two or more parts, and may perform calibration as needed. In this case, the processor 110 may monitor the positions of the two or more parts using the internal control values of the medical robot along with coordinate interpretation based on the images acquired through the camera 130.

Referring to FIG. 5, the processor 110 may identify two or more parts that are control targets based on an image acquired through the camera 130 in step S310. The processor 110 may determine the presence or absence of markers marked on the parts based on the image acquired through the camera 130 in step S320. In this case, the markers may be understood as portions of the parts of the medical robot or representations marked in the space where the parts are actually present. For example, the markers may be marked in the form of points on corners of the top surfaces of the parts. The processor 110 may estimate feature points from the image acquired through the camera 130 based on a different algorithm depending on the presence or absence of such markers.

When it is determined that markers are present, the processor 110 may search for feature points to be estimated for the coordinate interpretation of the image based on the markers in step S331. In other words, the processor 110 may determine the markers shown in the image as the feature points to be estimated for the coordinate interpretation of the image. When the markers are estimated as the feature points in this manner, the processor 110 may determine whether the two or more parts that are control targets have moved to the target positions in step S340. When the target parts have moved to the target positions, the processor 110 may perform the above-described marker presence/absence determination and feature point search computations again. That is, the processor 110 may acquire an image again through the camera 130 and determines the presence or absence of markers in step S350, and may perform a feature point search computation when it is determined that markers are present in step S361. However, when it is determined through step S320 that markers are present before the parts move to the target positions, step S350 may be omitted. For example, the processor 110 may estimate markers, present in a first image acquired at a time point (hereinafter referred to as a first time point) before the target parts move to the target positions, as first feature points. The processor 110 may estimate markers, present in a second image acquired at a time point (hereinafter referred to as a second time point) after the target parts have moved to the target positions, as second feature points.

When the feature point search before and after the movement of the target parts is completed, the processor 110 may match the feature points found before the movement of the target parts against the feature points found after the movement of the target parts in step S370. The processor 110 may estimate a matrix for the geometric transformation of the images acquired before and after the movement of the target parts by using the results of the matching in step S380. Thereafter, the processor 110 may estimate the coordinate differences between the target parts after the target parts have moved to the target positions based on the matrix in step S390. For example, the processor 110 may match the first feature points based on the first time point against the second feature points based on the second time point. Through this matching computation, the processor 110 may analyze the correlation between the images before and after the movement of the target parts, and may determine the coordinate relationship according to changes before and after the movement of the target parts. The processor 110 may estimate an affine matrix using the mutually matched feature points. In this case, the processor 110 may exclude outliers using a technique such as the least square method, the random sample consensus (RANSAC), and/or the like. The processor 110 may estimate the relative positions of the target parts at the second time point based on the relative positions of the target parts at the first time point, determined through the feature point matching, and the affine matrix.

When it is determined that the markers are not present, the processor 110 may extract feature points, to be estimated for coordinate interpretation, from the image acquired through the camera 130 in step S332. Since the markers are not present, the processor 110 may extract feature points for the parts by analyzing the structural features of the medical robot shown in the image. Once the feature point extraction has been completed, the processor 110 may compute feature descriptors based on the feature points in step S333. The processor 110 may compute feature descriptors having scale and rotation invariant characteristics through the scale invariant feature transform (SIFT) algorithm, the speed-up robust feature (SURF) algorithm, the oriented fast and rotated brief (ORB) algorithm, and/or the like. Once the feature point extraction and the feature descriptor estimation have been completed, the processor 110 may determine whether the two or more parts that are control targets have moved to the target positions in step S340. When the target parts have moved to the target positions, the processor 110 may perform the above-described marker presence/absence determination and feature descriptor estimation computations again. That is, the processor 110 may acquire an image again through the camera 130 and determine the presence or absence of markers in step S350, may perform computation for the extraction of feature points when it is determined that markers are not present in step S362, and may perform computation for the estimation of feature descriptor in step S363. However, when it is determined through step S320 that markers are not present before the parts move to the target positions, step S350 may be omitted. For example, the processor 110 may estimate first feature points and first feature descriptors based on a first image acquired at a time point (hereinafter referred to as a first time point) before the target parts move to the target positions. The processor 110 may estimate second feature points and second feature descriptors based on a second image acquired at a time point (hereinafter referred to as a second time point) after the target parts have moved to the target positions.

When feature point £ extraction and feature descriptor computations are completed before and after the movement of the target parts, the processor 110 may match the feature points found before the movement of the target parts against the feature points found after the movement of the target parts in step S370. In this case, the processor 110 may match the feature points by utilizing the feature descriptors computed before and after the movement of the target parts. The processor 110 may estimate a matrix for the geometric transformation of the images acquired before and after the movement of the target parts by using the results of the matching in step S380. Thereafter, the processor 110 may estimate the coordinate differences between the target parts after the target parts have moved to the target positions based on the matrix in step S390. For example, the processor 110 may match the first feature points based on the first time point against the second feature points based on the second time point. In this case, the processor 110 may use the first feature descriptors and the second feature descriptors to match the first feature points against the second feature points. Through this matching computation, the processor 110 may analyze the correlation between the images before and after the movement of the target parts, and may determine the coordinate relationship according to changes before and after the movement of the target parts. The processor 110 may estimate an affine matrix using the mutually matched feature points. In this case, the processor 110 may exclude outliers by using a technique such as the least square method, the random sample matching method, and/or the like. The processor 110 may estimate the relative positions of the target parts at the second time point based on the relative positions of the target parts at the first time point, determined through the feature point matching, and the affine matrix.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as unitary may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

1. A method for monitoring control of a medical robot, the method being performed by a computing device including at least one processor, the method comprising:
generating first position data indicating relative positions between two or more parts included in a medical robot based on an image acquired through a camera;
generating second position data indicating relative positions between the parts based on control data intended to move the parts to target positions;
computing an error between the first position data and the second position data; and
monitoring results of movements of the parts to the target positions based on the error.

2. The method of claim 1, wherein generating the first position data indicating the relative positions between the two or more parts included in the medical robot based on the image acquired through the camera comprises:
estimating feature points for the parts based on the image acquired through the camera; and
generating the first position data based on the estimated feature points.

3. The method of claim 2, wherein the feature points are present on a same plane of the image and constitute three or more pairs.

4. The method of claim 2, wherein estimating the feature points for the parts based on the image acquired through the camera comprises:
estimating first feature points for the parts based on a first image acquired through the camera before the parts move to the target positions; and
estimating second feature points for the parts based on a second image acquired through the camera after the parts have moved to the target positions.

5. The method of claim 4, wherein generating the first position data based on the estimated feature points comprises:
matching the first feature points against the second feature points; and
generating position data indicating relative positions between the parts after the parts have moved to the target positions based on results of the matching.

6. The method of claim 5, wherein generating the position data indicating the relative positions between the parts after the parts have moved to the target positions based on the results of the matching comprises:
estimating a matrix for geometric transformation of the first and second images using the results of the matching; and
estimating coordinate differences between the parts after the parts have moved to the target positions based on the matrix.

7. The method of claim 2, further comprising determining whether markers marked on the parts are present based on the image acquired through the camera.

8. The method of claim 7, wherein, when it is determined that markers marked on the parts are present, the feature points correspond to the markers.

9. The method of claim 7, wherein, when it is determined that the markers marked on the parts are not present, estimating the feature points for the parts based on the image acquired through the camera comprises:
extracting the feature points from the image; and
computing feature descriptors based on the feature points.

10. The method of claim 7, wherein, when it is determined that the markers marked on the parts are not present, generating the first position data based on the estimated feature points comprises:
matching first feature points extracted based on a time point before the parts move to the target positions against second feature points extracted based on a time point after the parts have moved to the target positions based on the feature descriptors.

11. The method of claim 1, wherein the control data includes at least one of coordinate values generated by a processor of the medical robot to move the parts to the target positions, sensor values adapted for detection of positions of the parts, and encoder values of a motor intended for movements of the parts.

12. The method of claim 1, wherein monitoring the results of the movements of the parts to the target positions based on the error comprises:
estimating positional accuracy of the parts for the target positions by comparing the error with a reference value.

13. The method of claim 1, wherein monitoring the results of the movements of the parts to the target positions based on the error comprises:
determining whether calibration is required for at least one of positions of the parts by comparing the error with a reference value.

14. The method of claim 13, wherein monitoring the results of the movements of the parts to the target positions based on the error comprises:
when it is determined that the error exceeds the reference value, determining whether control of at least one of the parts requiring calibration is possible.

15. The method of claim 14, wherein monitoring the results of the movements of the parts to the target positions based on the error comprises:
when it is determined that control of at least one of the parts requiring calibration is impossible or that the error cannot be calibrated within a reference value through control, generating an alarm indicating that the calibration is required.

16. A computer program stored in a computer-readable storage medium, the computer program performing operations for monitoring control of a medical robot when executed on at least one processor,
wherein the operations comprise operations of:
generating first position data indicating relative positions between two or more parts included in a medical robot based on an image acquired through a camera;
generating second position data indicating relative positions between the parts based on control data intended to move the parts to target positions;
computing an error between the first position data and the second position data; and
monitoring results of movements of the parts to the target positions based on the error.

17. A computing device for monitoring control of a medical robot, the computing device comprising:
a processor including at least one core;
memory including program codes executable on the processor; and
a camera configured to photograph two or more parts included in a medical robot;
wherein the processor:
generates first position data indicating relative positions between two or more parts included in a medical robot based on an image acquired through a camera;
generates second position data indicating relative positions between the parts based on control data intended to move the parts to target positions;
computes an error between the first position data and the second position data; and
monitors results of movements of the parts to the target positions based on the error.
